(19) United States
(12) Patent Application Publication
(10) Pub. No.: US 2011/0269181 A1
(43) Pub. Date: Nov. 3, 2011

(54) METHOD AND SYSTEM FOR CONTINUOUS BIOTRANSFORMATION

(75) Inventors: CHEAN-YEH CHENG, Tao-Yuan (TW); HSIANG-RONG TSAI, Tao-Yuan (TW)

(73) Assignee: CHUNG YUAN CHRISTIAN UNIVERSITY, Tao-Yuan (TW)

(21) Appl. No.: 12/769,528

(22) Filed: Apr. 28, 2010

(51) Int. Cl. C12P 33/00; C12P 1/02; C12M 1/00; C12P 1/00

(52) U.S. Cl. 435/52; 435/41; 435/171; 435/289.1

(57) ABSTRACT

One embodiment of the present invention discloses a method for continuous biotransformation. The method is continuously supplying viable biocatalyst cells or biocatalyst biomolecules to a bioreactor containing substrate mediums, so as to mediate the substrate mediums to be converted into the desired bioproducts.

### BACKGROUND OF THE INVENTION

1. Field of the Invention

The present invention relates to methods and systems for continuous biotransformation with a high yield.

2. Description of Related Art

Estrogens and androgens are the important steroid hormones in animals to develop and maintain the reproductive system. β-Estradiol and testosterone are the most common estrogens and androgens, respectively, and widely applied for the birth control [1], the treatment of breast cancer [2], and the physiological replacement therapy for osteoporosis and chronic obstructive pulmonary disease [3,4].

As a result of the increasing concern of ecology, environmental protection, and production economy, the microbial technologies instead of the chemical syntheses are more and more utilized for the production of some industrially or pharmaceutically important chemicals recently. In 1937, Mamoli et al. reported the reduction of androstenedione to testosterone by Saccharomyces cerevisiae [5]. To date, even though many literatures indicated the 17β-reduction of 17-oxosteroid or vice versa can be carried out by different categories of microorganisms, yeast was still the favored biocatalyst for its regio- and stereo-selectivity [5]. The enzyme responsible for the reduction of estrone was classified as the 17β-hydroxysteroid dehydrogenase.

Since biotransformation was usually subject to the problems of substrate inhibition and/or product inhibition. To improve the productivity, the most common methods are the optimization of the process conditions such as temperature, pH, the stirring rate, and the oxygen level [6,7]. The immobilization of microbial cell was also used frequently to attack the problem [8,9]. Recently, the biphasic cell culture with organic and aqueous reaction medium are introduced to reduce both substrate and product inhibition and to enhance the yield and stereoselectivity for water insoluble substrate [10-12]. Also, an effective biotransformation of cholesterol performed under a cloud point system of the nonionic surfactant was reported in literature [13]. Singer et al. [14] have added various cyclodextrins to the batch cell culture and obtained better substrate conversion with less substrate and product inhibition for the reduction of androstenedione. In recent decades, the advancement of genetic engineering to clone the microorganism with a recombinant DNA was usually used for effective synthesis of the desired compounds [15].

Although the above mentioned methods can improve the product yield with batch type culture, some modified approaches in the reactor design such as fed-batch and semi-fed-batch cell culture are also developed to acquire the necessary efficiency and economy [16-18]. Evidences in reducing the substrate poison or the substrate inhibition by continuous cell culture have also been demonstrated in literatures [19-21]. However, most of the continuous cell cultures are performed with single reactor for the production of medical precursor or valuable enzyme and the yield is still not satisfied [22,23]. Therefore, it would be advantageous to provide a novel method and system for biotransformation, which can improve the product yield and enhance the product recovery within a short reaction period. (References: [1] Hill J W, Kolb D K. Chemistry for Changing Times. New Jersey: Pearson Education, Inc.; 2007. Chapter 19; [2] Sutherland T E, Anderson R L, Hughes R A, Altmann E, Schuliga M, Ziogas J, Stewart A. 2-Methoxyestradiol—a unique blend of activities generating a new class of anti-tumour/anti-inflammatory agents. Drug Discov Today 2007; 12:577-584; [3] Andersson T L G, Stehle B, Davidsson B, Höglund P. Drug concentration effect relationship of estradiol from two matrix transdermal delivery systems: Menorest® and Climara®. Maturitas 2000; 35:245-252; [4] Mazer N J. New clinical applications of transdermal testosterone delivery in men and women. J. Control. Release 2000; 65:303-315; [5] Donova M V, Egorova O V, Nikolayeva V M. Steroid 17β-reduction by microorganisms—a review. Process Biochem 2005; 40:2253-2262; [6] Berry H, Debat H, Larreta-Garde V. Excess substrate inhibition of soybean lipoxygenase-1 is mainly oxygen-dependent. FEBS Lett 1997; 408:324-326; [7] Mösche M, Jördening H-J. Comparison of different models of substrate and product inhibition in anaerobic digestion. Water Res 1999; 33:2545-2554; [8] Bekatorou A, Koutinas A A, Kaliafas A, Kanellaki M. Freeze-dried Saccharomyces cerevisiae cells immobilized on gluten pellets for glucose fermentation. Process Biochem 2001; 36:549-557; [9] Tsen J-H, Lin Y-P, King V A-E. Fermentation of banana media by using κ-carrageenan immobilized Lactobacillus acidophilus. Int J Food Microbiol 2004; 91:215-220; [10] Celik D, Bayraktar E, Mehmeto\check{g}lu Ü. Biotransformation of 2-phenylethanol to phenylacetaldehyde in a two-phase fed-batch system. Biochem Eng J 2004; 17:5-13; [11] Cheng C, Tsai H-R. Yeast-mediated enantioselective synthesis of chiral R-(+)- and S-(−)-1-phenyl-1-butanol from prochiral phenyl n-propyl ketone in hexane-water biphasic culture. J Chem Technol Biotechnol 2008; 83:1479-1485; [12] León R, Fernandes P, Pinheiro H M, Cabral J M S. Whole-cell biocatalysis in organic media. Enzyme Microb Technol 1998; 23:483-500; [13] Wang Z, Zhao F, Chen D, Li D. Cloud point system as a tool to improve the efficiency of biotransformation. Enzyme Microb Technol 2005; 36:589-594; [14] Singer Y, Shity H, Bar R. Microbial transformations in a cyclodextrin medium. Part 2. Reduction of androstenedione to testosterone by Saccharomyces cerevisiae. Appl Microbiol Biotechnol 1991; 35:731-737; [15] Lo C-K, Pan C-P, Liu W-H. Production of testosterone from phytosterol using a single-step microbial transformation by a mutant of Mycobacterium sp. J Ind Microbiol Biotechnol 2002; 28:280-283; [16] Cheng C, Ma J-H. Enantioselective synthesis of S-(−)-1-phenylethanol in Candida utilis semi-fed-batch cultures. Process Biochem 1996; 31:119-124; [17] Crolla A, Kennedy K J. Fed-batch production of citric acid by Candida lipolytica grown on n-paraffins. J Biotechnol 2004; 110:73-84; [18] Ding S, Tan T. L-lactic acid production by Lactobacillus casei fermentation using different fed-batch feeding strategies. Process Biochem 2006; 41:1451-1454; [19] Caravelli A H, Zaritzky N E. About the performance of Sphaerotilus natans to reduce hexavalent chromium in batch and continuous reactors. J Hazard Mater 2009; 168:1346-1358; [20] Magnusson L, Cicek N, Sparling R, Levin D. Continuous hydrogen production during fermentation of α-cellulose by the thermophilic bacterium Clostridium thermocellum. Biotechnol Bioeng 2009; 102:759-766; [21] Radniecki T S, Semprini L, Dolan M E. Expression of merA, trxA, amoA, and hao in continuously cultured Nitrosomonas europaea cells exposed to cadmium sulfate additions. Biotechnol Bioeng 2009; 104:1004-1011; [22] Chan E-C, Kuo J. Biotransformation of dicarboxylic acid by immobilized Cryptococcus cells. Enzyme Microb Technol 1997; 20:585-589; [23] Domingues L, Lima N, Teixeira J A. Aspergillus niger β-galactosidase production by yeast in a continuous high cell density reactor. Process Biochem 2005; 40:1151-1154.)

### SUMMARY OF THE INVENTION

An object of the present invention is to provide novel methods and systems for biotransformation, which can improve the product yield and enhance the product recovery within a short reaction period.

According to the object, one embodiment of the present invention provides a method for continuous biotransformation, comprising: continuously supplying viable biocatalyst cells or biocatalyst biomolecules to a bioreactor containing substrate mediums, so as to mediate the substrate mediums to be converted into the desired bioproducts.

According to the object, one embodiment of the present invention provides a method for continuous biotransformation, comprising: continuously supplying a yeast solution comprising viable yeast cells to at least one bioreactor with a first flow rate; continuously supplying a substrate solution containing substrate mediums to each of the bioreactor with a second flow rate, whereby the yeast cells mediate the substrate mediums to be converted into the desired microbial products and thus a product solution is formed; and continuously drawing the product solution containing the microbial products from each bioreactor with a third flow rate; wherein the third flow rate is substantially equal to the summation of the first flow rate and the second flow rate.

According to the object, one embodiment of the present invention provides a system for continuous biotransformation, comprising: a first tank for continuously supplying a yeast solution comprising viable yeast cells to a bioreactor with a first flow rate; a first reservoir for continuously supplying a substrate solution containing substrate mediums to the bioreactor with a second flow rate, whereby the yeast cells mediate the substrate mediums to be converted into the desired microbial products and a product solution is formed; and a first circulation device for continuously drawing the product solution containing the microbial products to a collection stage from the bioreactor with a third flow rate; wherein the third flow rate is equal to the summation of the first flow rate and the second flow rate.

### BRIEF DESCRIPTION OF THE DRAWINGS

FIG. 1 shows a method for continuous biotransformation according to one embodiment of the present invention.

FIG. 2 shows a system 10 for continuous biotransformation according to one embodiment of the present invention.

FIGS. 3A to 3D show results for different initial concentration of estrone in the reaction tank and different continuous feed rate of estrone.

FIGS. 4A to 4D show the results of estrone reduction with different estrone feed concentration and different draw rate of the product solution for the continuous cell culture of dual stirred tank system.

FIG. 5 shows the variations of the average concentrations for both estrone and β-estradiol in the reaction tank and the product culture collection flask.

### DESCRIPTION OF THE PREFERRED EMBODIMENT

Reference will now be made in detail to specific embodiments of the invention. Examples of these embodiments are illustrated in accompanying drawings. While the invention will be described in conjunction with these specific embodiments, it will be understood that it is not intended to limit the invention to these embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well-known process operations and components are not been described in detail in order not to unnecessarily obscure the present invention. While drawings are illustrated in details, it is appreciated that the quantity of the disclosed components may be greater or less than that disclosed, except expressly restricting the amount of the components.

The present disclosure describes method and system for biotransformation. Several specific details of the disclosure are set forth in the following description and drawings to provide a thorough understanding of certain embodiments of the disclosure. One skilled in the art, however, will understand that the present disclosure may have additional embodiments, and that other embodiments of the disclosure may be practiced without several of the specific features described below.

One embodiment of the present invention provides a method for continuous biotransformation, comprising: continuously supplying viable biocatalyst cells or biocatalyst biomolecules to a bioreactor containing substrate mediums to mediate the substrate mediums to be converted into the desired bioproducts.

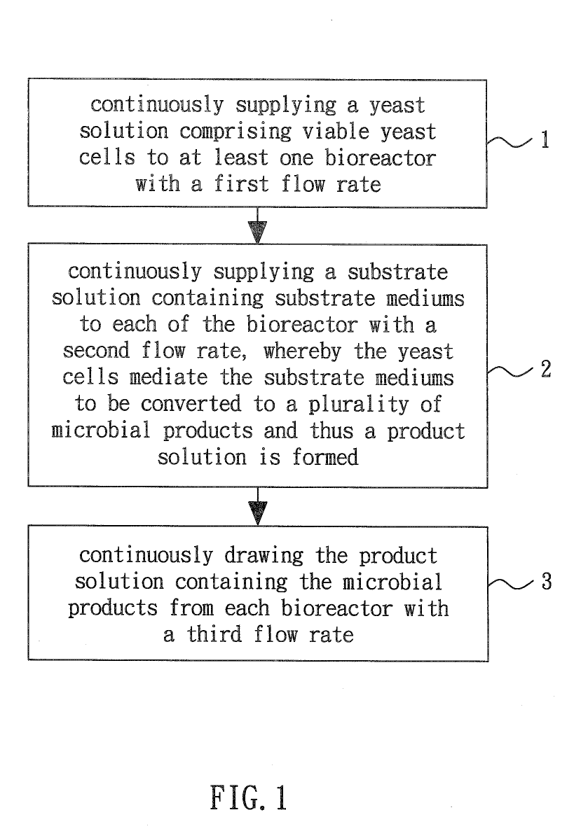

FIG. 1 shows a method for continuous biotransformation according to one embodiment of the present invention. The method comprising the steps of: step 1, continuously supplying a yeast solution comprising viable yeast cells to at least one bioreactor with a first flow rate; step 2, continuously supplying a substrate solution containing substrate mediums to each of the bioreactor with a second flow rate, such that the yeast cells mediate the substrate mediums to be converted into the desired microbial products and thus a product solution is formed; and step 3, continuously drawing the product solution containing the microbial products from each bioreactor with a third flow rate, where the third flow rate is substantially equal to the summation of the first flow rate and the second flow rate

In addition, in the method described in FIG. 1 the yeast solution may be fed from a stirred tank, incubation mediums may be fed to the stirred tank with a fourth flow rate, yeast may be initially inoculated into the incubation mediums in the stirred tank, and air may be continuously supplied to the stirred tank to incubate the viable yeast cells and forms the yeast solution. In addition, the yeast cells may comprise Sacharomyces cerevisiae, the substrate mediums may comprise estrone or androstenedione, and the microbial products may comprise estradiol or testosterone.

The term “biomolecules” comprises monoclonal antibodies, polyclonal antibodies, nucleic acids, proteins, enzymes, lipid, polysaccharides, sugars, peptides, polypeptides and bioligands.

The term “bioreactor”, as used in the present context, refers to a space, where a biotransformation can take place. The term “bioreactor” comprises any device allowing a contact of a solution to be converted with a biocatalyst or yeast, like a stirred tank (reactor), a fluidized bed reactor, a batch reactor, a plug flow reactor, a filter reactor, a membrane filter reactor, or a ceramics filter reactor. It is also possible to use a combination of two or more of the mentioned reactor types as a bioreactor. A single vessel containing a high number of defined flow paths where biocatalytic conversion can take place is still defined as one bioreactor.

The following exemplary embodiment discloses a system consisting of a new type of continuous cell culture with dual stirred tank connected in series to perform the continuous reduction of estrone in which the reduction of estrone was catalyzed by yeast S. cerevisiae. One skilled in the art understands that the system of the present invent can be applied to produce other microbial products such as testosterone except the reduction of estrone and other yeasts can be employed.

In addition, two comparative examples respectively employ “batch type cell culture” and “continuous cell culture with single stirred tank” to reduce the estrone in a same condition for comparing the yield.

### Culture Media—Prepare the Yeast Solution

Yeast (Saccharomyces cerevisiae) grew on agar slants was inoculated into 100 mL of the following incubation medium: 1.5 g KH₂PO₄, 2.9 g K₂HPO₄, 1.3 g (NH₄)₂SO₄, 1.8 g MgSO₄.7H₂O, 0.0175 g CaCl₂.2H₂O, 0.1 mL (1.25%, w/v) FeSO₄, 20.0 g D-(+)-glucose, and 1.0 L distilled water. The cell growing procedure and conditions are the same as mentioned in: Cheng C, Tsai H-R. Yeast-mediated enantioselective synthesis of chiral R-(+)- and S-(−)-1-phenyl-1-butanol from prochiral phenyl n-propyl ketone in hexane-water biphasic culture. J Chem Technol Biotechnol 2008; 83:1479-1485, the entire disclosure of which is incorporated herein by reference. The yeast-inoculated incubation mediums are then vented or air-bubbled to incubate the viable yeast cells and thus form the yeast solution.

### COMPARATIVE EXAMPLE

### Batch Type Cell Culture

The yeast mediated 17-oxosteroid (in this example, estrone) batch reduction uses the same incubation mediums as mentioned above to grow the yeast cells in an automatic control 2-L mini-jar fermentor (Eyela Model M-100, Tokyo, Japan) or in a 3-L stirred tank fermentor (BTF-A3L, Bio-Top Corporation, Taichung, Taiwan). After two days of the yeast cell growth, the estrone dissolved in 5.0 mL absolute ethanol is directly added into the yeast cell grown culture to start the reaction. The reduction is controlled at pH 5.0, 30° C., a stirring rate at 150 rpm, and without air bubbling. In general, the reaction period is six days. The cell culture sampled every 24 hours is filtered for further analysis.

### COMPARATIVE EXAMPLE

### Continuous Cell Culture with Single Stirred Tank

Yeast (Saccharomyces cerevisiae) cell is first grown in a stirred tank containing the above same incubation mediums for two days. Then 5.4 mg estrone dissolved in 5.0 mL absolute ethanol is directly added to the cell culture. Subsequently, 35 mg L⁻¹ estrone at 0.1 or 0 2 mL min⁻¹ was continuously fed to the cell culture and the cell culture is continuously drawn to a collection flask at the same rate. The cell culture is reacted with conditions of pH 5.0, 30° C., stirring rate 150 rpm, and without air bubbling. The cell culture collected in the flask is sampled, filtered, and analyzed every eight hours in the first day and every twenty-four hours for the rest of the reaction period. A new empty flask is used for the cell culture collection at each sampling time.

### Preferred Embodiment—Continuous Cell Culture of Dual Stirred Tank

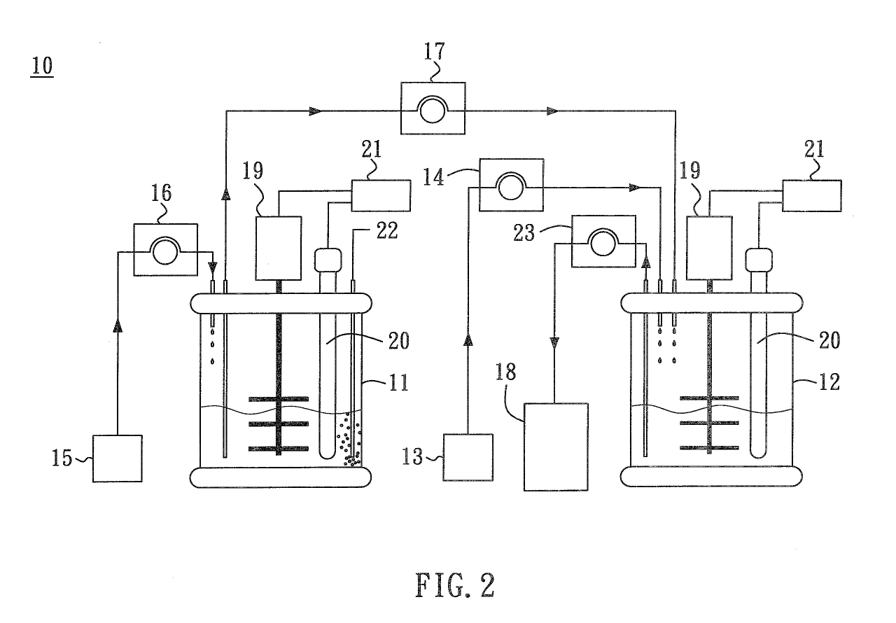

FIG. 2 shows a system 10 for continuous biotransformation according to one embodiment of the present invention, which exemplarily shows a continuous cell culture of dual stirred tank for estrone reduction. Two stirred tanks (11/12) connected in series are used in this example that one tank 11 is for the cell incubation and the other tank 12 is for the biotransformation. Both two stirred tanks 11/12 are equipped with a motor 19 for stirring the contents of the tank 11/12 and a PH meter 20 for controlling the pH of the contents at a predetermined value, and both the motor 19 and the pH meter 20 are controlled by a controller 21. A pretreatment procedure may be performed before the biotransformation is started. The pretreatment procedure comprises to grow yeast (Saccharomyces cerevisiae) in both stirred tanks (11/12) containing the above same incubation mediums with conditions of about pH 7.0, 30° C., stirring rate 150 rpm, and air-vented. After that, the conditions of the cell incubation stirred tank 11 are kept unchanged, and the conditions of the reaction stirred tank 12 will be changed.

Then zero or a measured amount of estrone dissolved in 5.0 mL absolute ethanol is directly added into the 2-L reaction stirred tank 12, which is controlled at about pH 5.0, 30° C., stirring rate 150 rpm, and without air bubbling. Subsequently, a certain amount of substrate medium 13 (estrone) is continuously fed to the reaction stirred tank 12 at a flow rate of 0.1 mL min⁻¹ with a circulation device 14, such as a peristaltic pump (Longerpump™, BT50-1J, Baoding, Hebei, China). In the meantime, fresh incubation medium 15 is continuously added to the 3-L cell incubation tank 11 via another circulation device 16. Air is continuously provided via an air inlet 22 to continuously incubate the viable yeast cells and forms the yeast solution in the cell incubation tank 11. The yeast solution 15 comprising viable yeast cells is continuously fed to the reaction stirred tank 12 via another circulation device 17, such as a two channel peristaltic pump (Longerpump™ BT100-2J) at a flow rate of 0.15 mL min⁻¹.

In the reaction stirred tank 12, yeast cells (Saccharomyces cerevisiae) mediate the substrate mediums (estrone) to be converted to the desired microbial products (β-estradiol) and forms a product solution, which comprises the microbial products, unconsumed yeast cells, unconsumed incubation medium, and the likes, and which is drawn continuously with another circulation device 23, such as a Longerpump™ peristaltic pump (BT50-1J), at a flow rate of 0.25 mL min⁻¹ to a collection stage 18, such as a collection flask, such that the liquid volume of the reaction stirred tank 12 can be kept at one liter. During the reaction, product solution drawn from the reaction cell culture is monitored by HPLC. The sampling time is the same as the above section and a new empty flask was used at each sampling time.

The microbial products can be obtained from the product solution via a separating process, such as filtration. Note that in another embodiment of the present invention, the viable yeast cells may be also separated from the product solution in the collection stage 18 and recycled to the cell incubation stirred tank 11 via another circulation device (not shown). Also note that in another embodiment the system may comprise two or more reaction stirred tanks 12 that are parallel connected.

### Cell Mass Measurement

Ten milliliters cell culture, i.e., product solution, is sampled at regular reaction time interval from the batch-type cell culture or separately from the reaction tank 12 of the continuous dual stirred tank system. The yeast solution is also sampled from the cell incubation tank 11 to investigate the cell mass. The sampled cell culture is filtered through the micro-porous membrane filter (mixed cellulose esters, 47 mm diameter and 0.2 μm pore size, Advantec MFS Inc., California, USA). The filtered cell on the membrane is dried in the oven at 50° C. for about 24 hours. The dry cell mass is obtained by deduction the weight of the original empty membrane.

### Substrate and Microbial Product Analysis

Two milliliters cell culture is sampled from the batch-type cell culture and from both stirred tank and the product collection flask of the continuous dual stirred tank system at the time interval described before. The cell culture sample is filtered by the disposable syringe filter of polyvinylidene fluoride (PVDF) membrane (Millex® HV, 13 mm diameter and 0.45 μm pore size, Millipore, Mass., USA). The filtrate is then analyzed by the on-line solid-phase extraction (SPE) coupled HPLC (JASCO PU1580, Tokyo, Japan) and an ultraviolet detector (Shimadzu SPD-10A, Kyoto, Japan). The SPE cartridge (25 mm×4 mm LiChrospher® RP-18e ADS, Merck) and the analytical column (100 mm×4.6 mm Chromolith™ Performance RP-18e column, Merck) are connected parallel through a six-port switching valve. For estrogen analysis, the mobile phases delivered through the SPE cartridge and the analytical column are a mixture of acetonitrile and water at a volume ratio of 1:9 and 1:3, respectively. The flow rate of mobile phase for elution through the SPE cartridge and the analytical column was at 0.5 and 3.0 mL min⁻¹, respectively.

### Diastereomeric Excess

Since 17β-estradiol is the corresponding epimer of estrone, the diastereomeric excess value (% d.e.) is used for expressing the reaction stereoselectivity instead of the enantiomeric excess value (% e.e.). The % d.e. is calculated by the following formula:

$$\% d . e . = \frac{moles of β \text{-} epimer - moles of α \text{-} epimer}{total moles of α \text{-} epimer and β \text{-} epimer} \times 100 \% \qquad ( 1 )$$

### Results—Optimal Condition of Batch Type Cell Culture for Estrone Reduction

For a batch type cell culture estrone is used as the model compound to search for optimal reaction conditions. With an initial 12.0 mg estrone in the fermentor the reaction is operated as described previously but with different pH values (4.0, 5.0, 6.0, and 7.0). For all experiments the cell mass of the cell culture continuously decreased during the reaction period. Since a maximum yield of 42.5% is found for the cell culture operated at pH 5.0, pH 5.0 is selected to use for the rest of experiments of the S. cerevisiae mediated reduction. Only β-estradiol is found for all batch type cell cultures that indicated an excellent regio- and stereo-selective reduction of estrone.

In order to find the optimal initial substrate concentration for an acceptable yield and diastereomeric excess value, different initial estrone concentrations of 2.7, 5.4, 12.0, and 135 mg L⁻¹ are tested and the results are shown in Table 1. When an initial concentration of 5.4 mg L⁻¹ estrone is used for a six-day reaction period a maximal β-estradiol production yield of 54.8% and a recovery of 3.0 mg are obtained at the third day. Since β-estradiol is the only product in all cell cultures the diastereomeric excess (% d.e.) calculated is larger than 99% and indicated an excellent yeast mediated stereoselectivity.

TABLE 1
Summary of the results for batch and continuous cell culture reduction.

| | | | | | |
|---|---|---|---|---|---|
| Initial | Substrate | Reaction cell | Maximal | Maximal | |
| substrate | feed | culture draw | product | accumulated | Maximal |
| concentration | concentration | rate^{a} | yield^{b} | product^{b} | % d.e.^{b} |
| (mg L⁻¹) | (mg L⁻¹) | (mL min⁻¹) | (%) | (mg) | (%) |

Batch cell culture
Estrone

| | | | | | | | |
|---|---|---|---|---|---|---|---|
| 2.7 | — | — | 42.0 | (2nd d) | 1.1 | (2nd d) | >99 |
| 5.4 | — | — | 54.8 | (3rd d) | 3.0 | (3rd d) | >99 |
| 12.0 | — | — | 42.5 | (5th d) | 5.1 | (5th d) | >99 |
| 135.0 | — | — | 26.8 | (5th d) | 36.4 | (5th d) | >99 |

Continuous cell culture of single stirred tank

| | | | | | | | |
|---|---|---|---|---|---|---|---|
| 5.4 | 35.0 | 0.20 | 33.5 | (1st d) | 4.2 | (2nd d) | >99 |
| 5.4 | 35.0 | 0.10 | 24.5 | (1st d) | 4.0 | (4th d) | >99 |

Continuous cell culture of dual stirred tank system

| | | | | | | | |
|---|---|---|---|---|---|---|---|
| 0 | 45.0 | 0.25 | 40.9 | (2nd d) | 8.4 | (4th d) | >99 |
| 0 | 82.5 | 0.25 | 50.4 | (1st d) | 6.0 | (1st d) | >99 |
| 4.0 | 45.0 | 0.25 | 49.1 | (2nd d) | 10.0 | (3rd d) | >99 |
| 6.8 | 45.0 | 0.25 | 53.3 | (1st d) | 10.5 | (4th d) | >99 |
| 5.4 | 25.0 | 0.20 | 36.0 | (1st d) | 4.8 | (4th d) | >99 |
| 5.4 | 35.0 | 0.20 | 44.4 | (1st d) | 8.3 | (4th d) | >99 |
| 5.4 | 35.0 | 0.30 | 34.8 | (1st d) | 5.4 | (4th d) | >99 |
| 5.4 | 35.0 | 0.25 | 49.4 | (1st d) | 7.6 | (4th d) | >99 |
| 5.4 | 45.0^{c} | 0.25 | 64.8 | (2nd d) | 12.9 | (3rd d) | >99 |
| 5.4 | 50.0 | 0.25 | 46.6 | (2nd d) | 11.7 | (4th d) | >99 |

^{a}Draw rate = cell transfer rate + substrate feed rate (=0.1 mL min⁻¹)
^{b}The number in the parenthesis is the reaction day.
^{c}The average value of duplicate experiment.

It is also noted that in almost all experiments the concentration of β-estradiol decreases during the final reaction day. This phenomenon is probably due to the depletion of incubation mediums to cause cell death and broken-down and the released substances or enzymes consumed estrone and β-estradiol. Since the 17β-hydroxysteroid dehydrogenase is an intracellular enzyme and is active only within a viable cell, the estrone reduction to the desired β-estradiol can only proceed with a viable cell during the fermentation. Therefore, to keep a large amount of viable yeast cell is necessary to give a higher product yield within a short period.

Results of the Continuous Cell Culture with Single Stirred Tank

For the continuous cell culture, 5.4 mg estrone is initially added into the reaction culture and 35 mg L⁻¹ estronein the reaction medium is continuously fed to the fermentor at a flow rate of either 0.2 or 0.1 mL min⁻¹ and the fermentation culture is drawn at the same flow rate. The total amount of β-estradiol recovered from the continuous cell culture is 4.2 mg at the drawing rate of 0.2 mL min⁻¹ for two days and is 4.0 mg at the draw rate of 0.1 mL min⁻¹ for four days. However, the overall yields for these two continuous cell cultures are less than 34% as shown in Table 1 due to a large decrease of the cell mass in the fermentor by the continuous draw of the cell culture.

### Results of the Continuous Cell Culture of Dual Stirred Tank

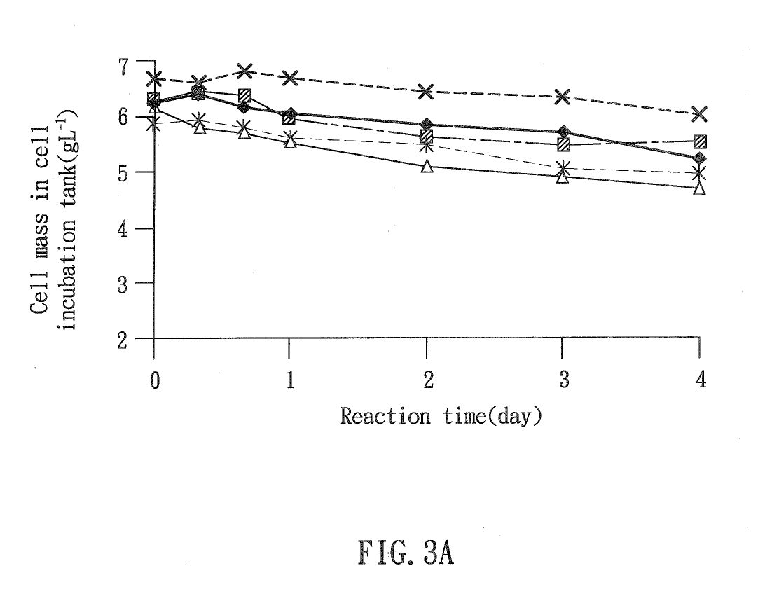

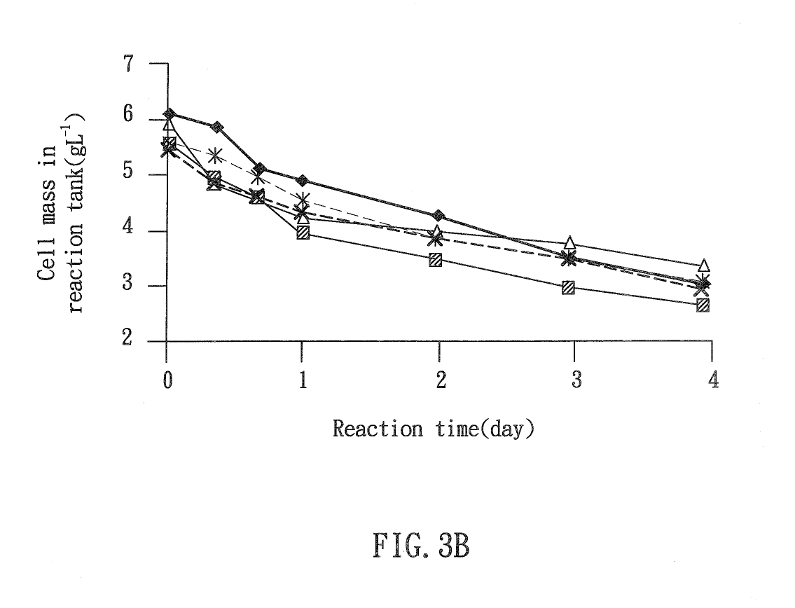

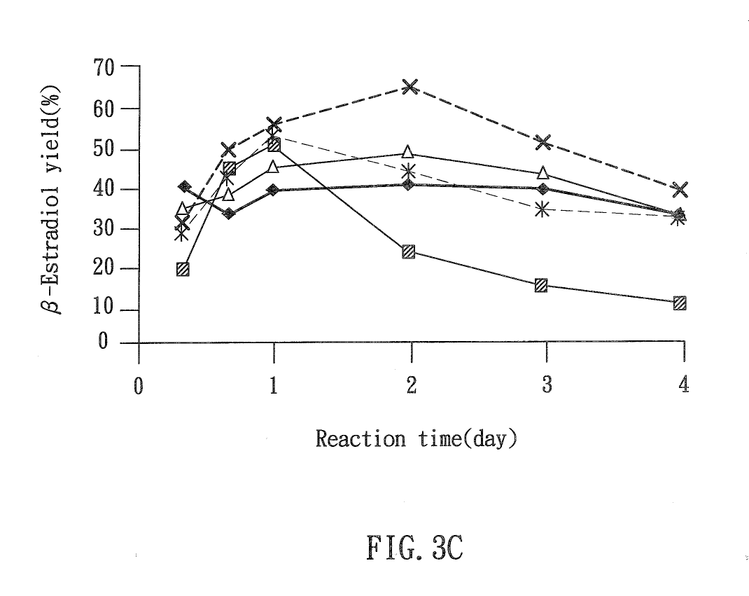

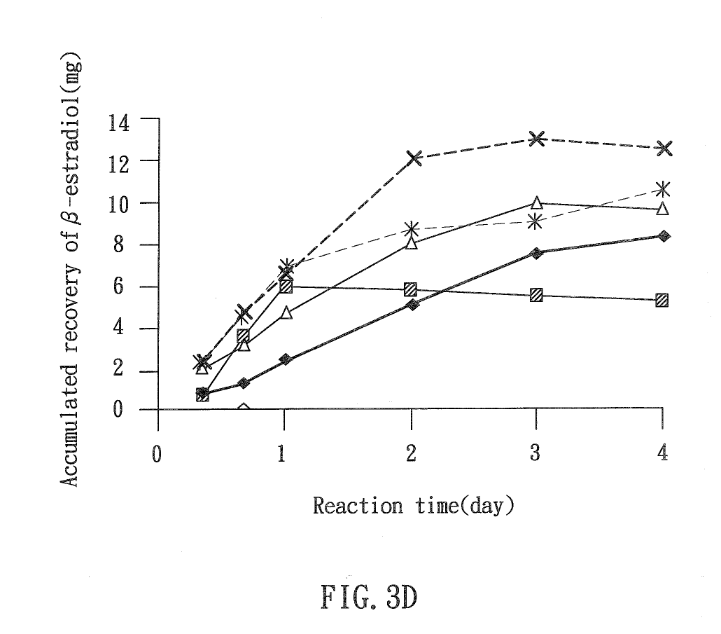

In order to maintain a large amount of viable cells to perform the desired estrone reduction and reduce the substrate inhibition, the continuous cell culture with dual stirred tank in series is designed to reach the goals. Since the cell activity and cell mass kept decrease during the reaction, to utilize the high cell activity at the initial few days some estrone are added at the start of reaction. The results for different initial concentration of estrone in the reaction tank and different continuous feed rate of estrone are shown in FIGS. 3A to 3D and in which FIG. 3A denotes the variation of cell mass in the cell incubation stirred tank; FIG. 3B the variation of cell mass in the reaction stirred tank; FIG. 3C the β-estradiol yield of the estrone reduction, FIG. 3D the accumulated recovery of β-estradiol; ♦, 0 mg initial estrone and continuous feed of 45 mg L⁻¹ estrone; ▪, 0 mg initial estrone and continuous feed of 82.5 mg L⁻¹ estrone; ▴, 4.0 mg initial estrone and continuous feed of 45 mg L⁻¹ estrone; ×, the average of duplicate experiment with 5.4 mg initial estrone and continuous feed of 45 mg L⁻¹ estrone; *, 6.8 mg initial estrone and continuous feed of 45 mg L⁻¹ estrone.

For all experiments the cell mass in the cell incubation stirred tank decreased (FIG. 3A) even if fresh medium is continuously added due to the cell death and the continuous draw of the cell mass. The continuous supplying of yeast cells improves the decrease of cell mass (FIG. 3B) in the reaction stirred tank as compared with the batch cell culture. The line diagrams for the yield and the accumulated recovery of β-estradiol (FIGS. 3C and 3D) indicate that the appropriate initial amount of estrone is 5.4 mg with a continuous feed of 45 mg L⁻¹ estrone.

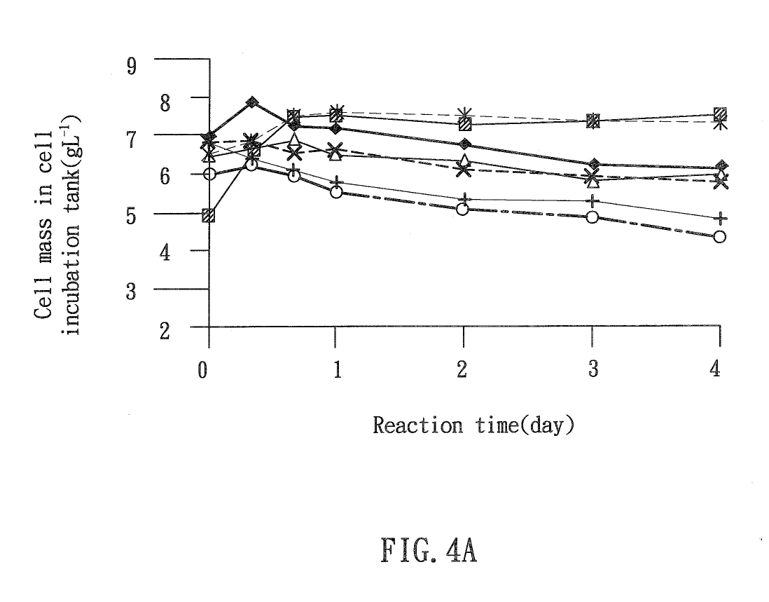

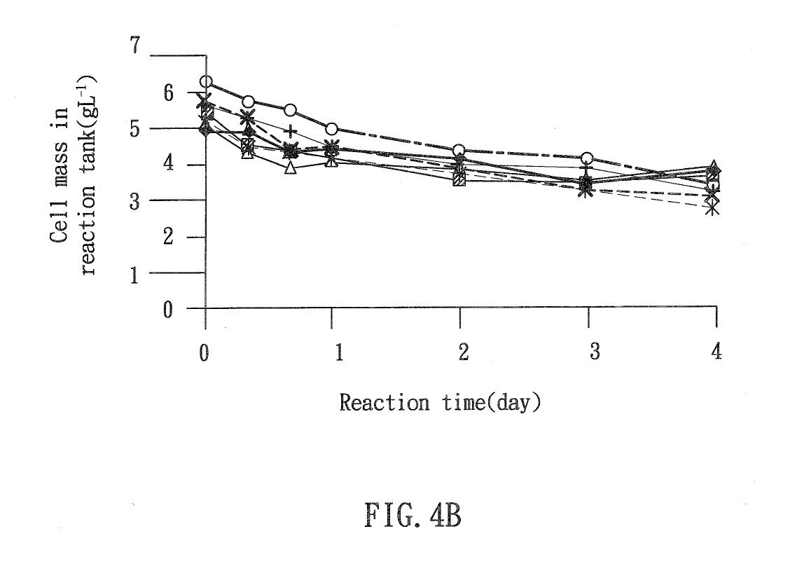

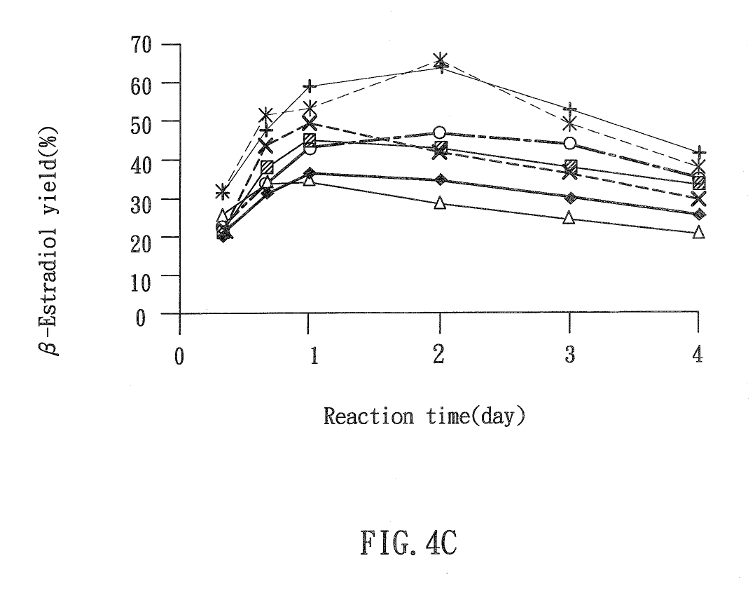

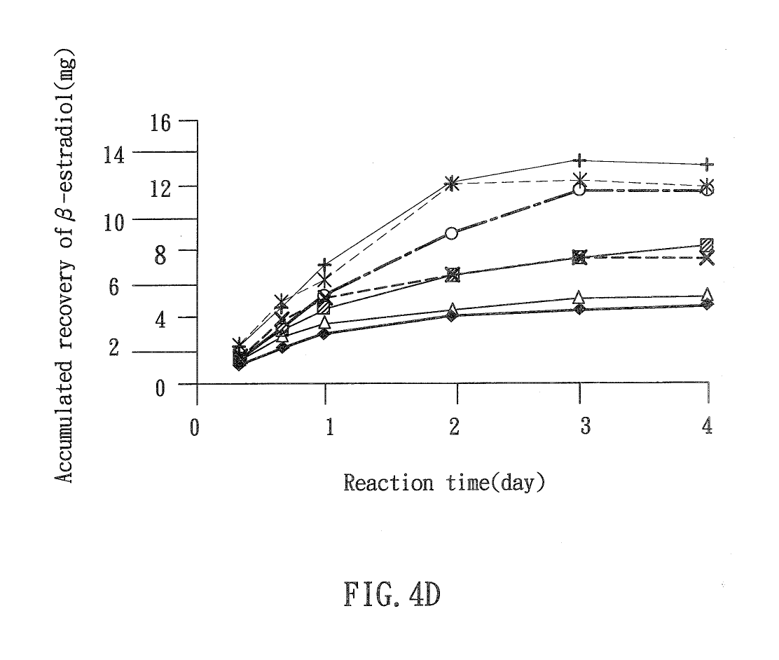

FIGS. 4A to 4D shows the results of estrone reduction with different estrone feed concentration and different draw rate (of the product solution) for the continuous cell culture of dual stirred tank system in which the initial amount of estrone in the reaction stirred tank is 5.4 mg, the estrone feed rate 0.1 mL min⁻¹, and in which FIG. 4A denotes the variation of cell mass in the cell incubation stirred tank; FIG. 4B the variation of cell mass in the reaction stirred tank; FIG. 4C the β-estradiol yield of the estrone reduction, FIG. 4D the accumulated recovery of β-estradiol; ♦, the continuous feed of 25 mg L⁻¹estrone and the reaction cell culture draw rate of 0.2 mL min⁻¹; ▪, the continuous feed of 35 mg L⁻¹ estrone and the reaction cell culture draw rate of 0.2 mL min⁻¹; ▴, the continuous feed of 35 mg L⁻¹ estrone and the reaction cell culture draw rate of 0.3 mL min⁻¹; ×, the continuous feed of 35 mg L⁻¹ estroneand the reaction cell culture draw rate of 0.25 mL min⁻¹; *, the first experiment with the continuous feed of 45 mg L⁻¹ estrone and the reaction cell culture draw rate of 0.25 mL min⁻¹; +, the second experiment with the continuous feed of 45 mg L⁻¹ estroneand the reaction cell culture draw rate of 0.25 mL min⁻¹; , the continuous feed of 50 mg L⁻¹ estrone and the reaction cell culture draw rate of 0.25 mL min⁻¹.

The estrone concentration of the continuous feed and the continuous draw rate of the reaction culture (product solution) are also optimized for the continuous cell culture of dual stirred tank. To determine the optimal estrone concentration in the feed, the continuous feed rate is fixed at 0.1 mL min⁻¹, the estrone concentration in the feed is varied from 35 to 50 mg L⁻¹, and the draw rate is varied from 0.2 to 0.3 mL min⁻¹ for comparison. As usual the cell mass in the incubation and reaction stirred tank decreased in most experiments (FIGS. 4a and 4b). The results show that a best yield of 65.5% at the second reaction day (FIG. 4c) and a product recovery of 12.3 mg β-estradiol at the third reaction day (FIG. 4d) are obtained for the experiment with a continuous estrone feed concentration of 45 mg L⁻¹ and a continuous reaction cell culture draw rate at 0.25 mL min⁻¹. This yield is significantly larger than the yield (54.8%) of the batch cell culture at the fifth day. A repeated experiment for this run also show a high yield of 64.1% at the second reaction day and a product recovery of 13.5 mg β-estradiol at the third reaction day. The average yield and the average accumulated recovery of β-estradiol are summarized in Table 1.

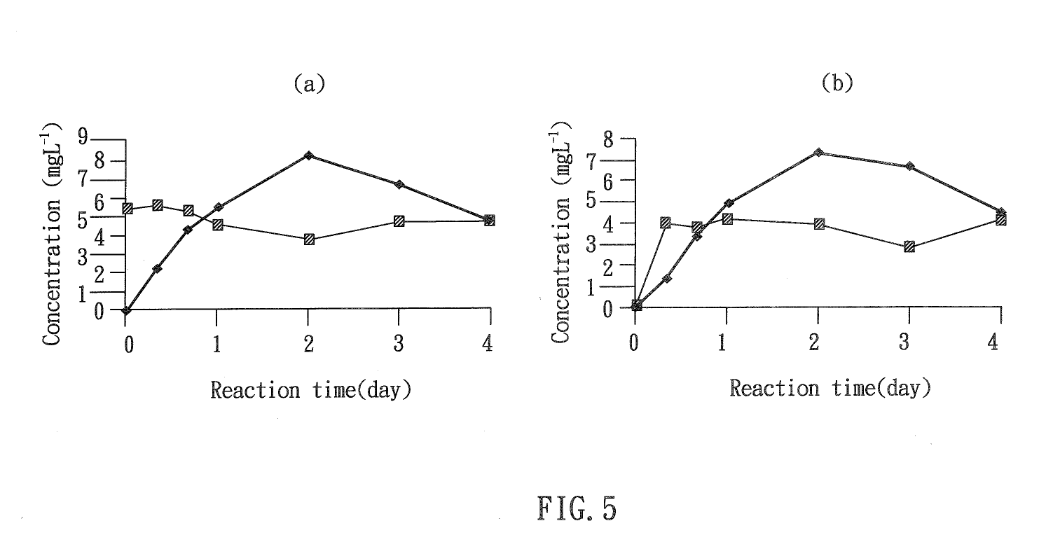

FIG. 5 shows the variations of the average concentrations for both estrone and β-estradiol in the reaction tank and the product culture collection flask in which the cell growth medium feed rate, the grown cell draw rate, and the reaction cell culture draw rate were the same as described in FIG. 4 and in which the initial amount of estrone in the reaction stirred tank is 5.4 mg with a continuous feed of 45 mg L⁻¹ estrone to the reaction tank at a rate of 0.1 mL min⁻¹ and in which (a) denotes the reaction stirred tank; (b) the cell culture collection flask; ▪, estrone; ♦, β-estradiol.

The estrone concentration in the reaction tank and the product collection flask during the reaction period is substantially steady and all lower than 5.6 mg L⁻¹ that indicates a stable reaction with low substrate inhibition. The production trend of β-estradiol in the reaction tank corresponded to the consumption trend of estrone. At the second day, the concentration of β-estradiol reached a maximum both in the reaction tank (FIG. 5a) and in the product collection flask (FIG. 5b). Since no α-estradiol is formed in the cell culture the stereo-selectivity for the continuous cell culture of dual stirred tank reduction of estrone to β-estradiol is excellent. Thus, the diastereomeric excess value (% d.e.) is >99%.

### Conclusions

New methods and systems for continuous biotransformation are successfully developed to solve the substrate inhibition and unknown consumption of the substrate and product. The developed continuous cell culture of dual stirred tank can maintain more viable cells in the reaction stirred tank than the batch cell culture by continuously supplying yeast cells from the incubation stirred tank. In the particular example for the estrone reduction, the β-estradiol yield efficiently increased and the accumulated recovery of β-estradiol increased 4.3-fold as compared to the batch cell culture. The stereo-selectivity of the yeast-mediated estrone reduction is larger than 99% d.e. according to the embodiments of the present invention.

Although specific embodiments have been illustrated and described, it will be appreciated by those skilled in the art that various modifications may be made without departing from the scope of the present invention, which is intended to be limited solely by the appended claims.

What is claimed is:

1. A method for continuous biotransformation, comprising: continuously supplying viable biocatalyst cells or biocatalyst biomolecules to a bioreactor containing substrate mediums, so as to mediate the substrate mediums to be converted into the desired bioproducts.

2. A method for continuous biotransformation, comprising:
continuously supplying a yeast solution comprising viable yeast cells to at least one bioreactor with a first flow rate;
continuously supplying a substrate solution containing substrate mediums to each of the bioreactor with a second flow rate, whereby the yeast cells mediate the substrate mediums to be converted into the desired microbial products and thus a product solution is formed; and
continuously drawing the product solution containing the microbial products from each bioreactor with a third flow rate.

3. The method as recited in claim 2, wherein the third flow rate is substantially equal to the summation of the first flow rate and the second flow rate.

4. The method as recited in claim 2, wherein the yeast solution is fed from a stirred tank, incubation mediums are fed to the stirred tank with a fourth flow rate, yeast are initially inoculated into the incubation mediums in the stirred tank, and air is continuously supplied to the stirred tank to incubate the viable yeast cells and forms the yeast solution.

5. The method as recited in claim 2, wherein the yeast cells comprise Saccharomyces cerevisiae.

6. The method as recited in claim 5, wherein the substrate mediums comprise estrone, and the microbial product comprises estradiol.

7. The method as recited in claim 2, wherein the substrate mediums comprise androstenedione, and the microbial product comprises testosterone.

8. The method as recited in claim 4, wherein the temperature of the solution in the cell incubation stirred tank is controlled at about 30° C., and the temperature of the solution in the bioreactor is controlled at about 30° C.

9. The method as recited in claim 4, wherein the pH value of the solution in the cell incubation stirred tank is controlled about 7, and the pH value of the solution in the bioreactor is controlled about 5.

10. A system for continuous biotransformation, comprising:
a first tank for continuously supplying a yeast solution comprising viable yeast cells to a bioreactor with a first flow rate;
a first reservoir for continuously supplying a substrate solution containing substrate mediums to the bioreactor with a second flow rate, whereby the yeast cells mediate the substrate mediums to be converted into the desired microbial products and a product solution is formed; and
a first circulation device for continuously drawing the product solution containing the microbial products to a collection stage from the bioreactor with a third flow rate.

11. The method as recited in claim 10, wherein the third flow rate is equal to the summation of the first flow rate and the second flow rate.

12. The system as recited in claim 10, wherein incubation mediums are fed to the first tank with a fourth flow rate, yeast are initially inoculated into the incubation mediums in the first tank, and air is continuously supplied to the first tank to incubate the viable yeast cells and forms the yeast solution.

13. The system as recited in claim 10, wherein the yeast cells comprise Saccharomyces cerevisiae.

14. The system as recited in claim 13, wherein the substrate mediums comprise estrone, and the microbial product comprises estradiol.

15. The system as recited in claim 14, wherein the yield of the estradiol is about 65% or more.

16. The system as recited in claim 14, wherein the reduction of estrone has a diastereomeric excess value (% d.e.) greater than 99%.

17. The system as recited in claim 14, wherein the estradiol is separated from the product solution in the collection stage.

18. The system as recited in claim 14, wherein viable yeast cells are separated from the product solution in the collection stage and recycled to the first tank.

19. The system as recited in claim 14, wherein the concentration of estradiol reaches a maximum value both in the bioreactor and in the collection stage after the biotransformation has proceeded for two days.

20. The system as recited in claim 14, wherein the substrate mediums comprise androstenedione, and the microbial product comprises testosterone.

21. The method as recited in claim 12, wherein the temperature of the solution in the first tank is controlled at about 30° C., and the temperature of the solution in the bioreactor is controlled at about 30° C.

22. The method as recited in claim 12, wherein the pH value of the solution in the first tank is controlled about 7, and the pH value of the solution in the bioreactor is controlled about 5.
